SEQUENCE LISTING (13 sequences)

SEQ ID NO: 1 - DNA, 91 bp, Artificial Sequence
  Description of Artificial Sequence Synthetic probe
ttgaactctgcttaaatccagtggtttttttttttttttatgttaagtgaccggcagcattttttttttttttttgaccgctgaagggctt

SEQ ID NO: 2 - DNA, 41 bp, Artificial Sequence
  Description of Artificial Sequence Synthetic oligonucleotide
  modified_base (25): a, c, g, t, unknown or other
ccactggatttaagcagagttcaanagcccttcagcggtca

SEQ ID NO: 3 - DNA, 47 bp, Artificial Sequence
  Description of Artificial Sequence Synthetic primer
aatttaatacgactcactatagggccactggatttaagcagagttca

SEQ ID NO: 4 - DNA, 15 bp, Artificial Sequence
  Description of Artificial Sequence Synthetic primer
  modified_base (1): biotin-t
tgaccgctgaagggc

SEQ ID NO: 5 - DNA, 41 bp, Artificial Sequence; identical to SEQ ID NO: 2
  Description of Artificial Sequence Synthetic oligonucleotide
  modified_base (25): a, c, g, t, unknown or other

SEQ ID NO: 6 - DNA, 47 bp, Artificial Sequence; identical to SEQ ID NO: 3
  Description of Artificial Sequence Synthetic primer

SEQ ID NO: 7 - DNA, 15 bp, Artificial Sequence
  Description of Artificial Sequence Synthetic primer
  modified_base (1): biotin-t
tgaccgctgaagggc

SEQ ID NO: 8 - DNA, 29 bp, Artificial Sequence
  Description of Artificial Sequence Synthetic oligonucleotide
tttttttttttttgaccgctgaagggctg

SEQ ID NO: 9 - DNA, 25 bp, Artificial Sequence
  Description of Artificial Sequence Synthetic oligonucleotide
tttttttttgaccgctgaagggctc

SEQ ID NO: 10 - DNA, 21 bp, Artificial Sequence
  Description of Artificial Sequence Synthetic oligonucleotide
tttttgaccgctgaagggcta

SEQ ID NO: 11 - DNA, 17 bp, Artificial Sequence
  Description of Artificial Sequence Synthetic oligonucleotide
tgaccgctgaagggctt

SEQ ID NO: 12 - RNA, 44 nt, Artificial Sequence
  Description of Artificial Sequence Synthetic oligonucleotide
  modified_base (28): a, u or g
gggccacuggauuuaagcagaguucaanagcccuucagcgguca

SEQ ID NO: 13 - DNA, 26 bp, Artificial Sequence
  Description of Artificial Sequence Synthetic oligonucleotide
ttgaactctgcttaaatccagtggct

(19) United States
(12) Patent Application Publication
(10) Pub. No.: US 2007/0225487 A1
(43) Pub. Date: Sep. 27, 2007

(54) Nucleic acid detection medium

(75) Inventors: Mats Bo Johan Nilsson, Uppsala (SE); Ulf Landegren, Uppsala (SE)

Correspondence Address: YOUNG & THOMPSON, 745 SOUTH 23RD STREET, 2ND FLOOR, ARLINGTON, VA, 22202, US

(73) Assignee: BIOCYCLICA AB, Sigtuna (SE)

(21) Appl. No.: 11/649,834

(22) Filed: Jan. 5, 2007

Related U.S. Application Data
(62) Division of application No. 10/257,280, filed on Feb. 3, 2003, now abandoned.

(30) Foreign Application Priority Data
Apr. 11, 2000 (EP) 00201301.9

(51) Int. Cl. C07H 21/00

(52) U.S. Cl. 536/25.3

(57) ABSTRACT

An optimum reaction medium for performing nucleic acid detection and a method employing the medium are scribed.

This invention relates to the detection of nucleic acids. More particularly the present invention relates to a medium in which the joining of nucleic acid oligonucleotides using DNA ligases and their subsequent detection is improved.

It is known in the prior art that it is important that RNA molecules representing members of gene families are distinguished in expression analyses, and even greater resolving power may be required to identify allelic variants of transcripts in order to investigate imprinting or to study the distribution of mutant genes in tissues. It is also important to be able to distinguish spliced or edited RNA variants, especially since it has become evident that there are many more human RNA species than genes in the genome.

Ligase-mediated gene detection has proven valuable for precise distinction of DNA sequence variants, but it is not known if ligases can also be used to distinguish variants of RNA sequences.

It is also known that the occurrence of specific RNA sequences can be measured to gauge the level of gene expression, either averaged over a tissue sample or according to the distribution of transcripts in tissue sections or in individual cells. Related RNA sequences can be distinguished by taking advantage of the reduced hybridisation stability of imperfectly matched hybridisation probes, but this can be problematic when many sequences are investigated under one set of hybridisation conditions, and when closely similar variants must be resolved. Since many genes are members of conserved gene families, this difficulty constitutes a significant problem in expression profiling, and the same holds for in situ analyses of related genes or allelic variants of single genes.

DNA ligases can be used to distinguish single-nucleotide variation in DNA sequences by taking advantage of the inefficient ligation of terminally mismatched oligonucleotides (Landegren, U., Kaiser, R., Sanders, J. & Hood, L. A ligase-mediated gene detection technique. Science 241, 1077-1080 (1988); Wu, D. Y. & Wallace, R. B. Specificity of the nick-closing activity of bacteriophage T4 DNA ligase. Gene 76, 245-254 (1989); Luo, J., Bergstrom, D. E. & Barany, F. Improving the fidelity of Thermus thermophilus DNA ligase. Nucleic Acids Res. 24, 3071-3078 (1996); Tong, J., Cao, W. & Barany, F. Biochemical properties of a high fidelity DNA ligase from Thermus species AK16D. Nucleic Acids Res. 27, 788-794 (1999)). It is also known that RNA molecules can template ligation of DNA oligonucleotides by T4 DNA ligase (Kleppe, K., van de Sande, J. H. & Khorana, H. G. Polynucleotide ligase-catalysed joining of deoxyribo-oligonucleotides on ribopolynucleotide templates and of ribo-oligonucleotides on deoxyribopolynucleotide templates. Proc. Natl. Acad. Sci. USA 67, 68-73 (1970); Fareed, G. C., Wilt, E. M. & Richardson, C. C. Enzymatic breakage and joining of deoxyribonucleic acid. VIII. Hybrids of ribo- and deoxyribonucleotide homopolymers as substrates for polynucleotide ligase of bacteriophage T4. J. Biol. Chem. 246, 925-932).

It is also known that some of the ATP dependent DNA ligases in eucaryotes and eucaryotic virus can use RNA as a template for DNA ligation (Tomkinson, A. E. and Mackey, Z. B. Structure and function of mammalian DNA ligases Mutation Res. 407 1-9 (1998),Sekiguchi, J. and Shuman, S. Ligation of RNA-containing duplexes by vaccinia DNA ligase Biochemistry 36 9073-9079 (1997),Sriskanda, V. and Shuman, S. Specificity and fidelity of strand joining by Chorella virus DNA ligase Nucleic Acids Res. 26 3536-3541 (1998)). However, it is not know how efficient the reaction is using these enzymes, and eucaryotic enzymes have not been used for gene analytic assays. Also ATP-dependent DNA ligases from thermophilic archeon such as Methanobacterium thermoautotrophicum (Sriskanda, V., et al. Characterization of an ATP-dependent DNA ligase from the thermophilic archaeon Methanobacterium thermoautotrophicum Nucleic Acids Res 28 2221-2228. (2000)) can join DNA oligonucleotides (data not shown). The general features of the detection medium described herein should be generally applicable for all DNA ligases.

RNA-templated ligation of DNA probes has been used to generate molecules, amplifiable by PCR via general sequences present at the remote ends of a pair of ligation probes (Hsuih, T. C. H. et al. Novel, ligation-dependent PCR assay for detection of hepatitis C virus in serum. J. Clin. Microbiol. 34, 501-507 (1996).). The method has been applied to detect viral RNA extracted from clinical and archival specimens with increased sensitivity compared to nested RT-PCR (Park, Y. N. et al. Detection of hepatitis C virus RNA using ligation-dependent polymerase chain reaction in formalin-fixed, paraffin-embedded liver tissues. Am. J Pathology 149, 1485-1491 (1996); Miyauchi, I., Moriyama, M., Zhang, D. Y. & Abe, K. Further study of hepatitis C virus RNA detection in formalin-fixed, paraffin-embedded liver tissues by ligation-dependent polymerase chain reaction. Path. Int. 48, 428-432 (1998)). RNA-templated ligation of RNA probes has been used for detection of transcripts in experiments where ligation products were amplified by the Qb replicase (Tyagi, S., Landegren, U., Tazi, M., Lizardi, P. M. & Kramer, F. R. Extremely sensitive, background-free gene detection using binary probes and Qb replicase. Proc. Natl. Acad. Sci. USA 93, 5395-5400 (1996).). RNA-templated ligation of either DNA or RNA probes can thus substitute for a reverse transcription (RT) step before amplification.

To date, no analysis has been presented of optimal reaction conditions for RNA-templated DNA ligation, and it is not known how well probe ligation reactions can discriminate among variants of RNA target sequences.

The present inventors have found that under low salt, particularly monovalent cations, and ATP conditions, high concentrations of T4 DNA ligase efficiently joined DNA oligonucleotides, hybridised in juxtaposition on RNA target strands.

Accordingly, the present invention provides a medium for the efficient ligation of oligonucleotides to target nucleic acid strands, the medium containing a low concentration of monovalent cations. For example the medium may contain low levels of sodium chloride. Preferably the medium contains less than 50 mMol monovalent cations, for example sodium chloride. More preferably, monovalent cations, for example sodium chloride, are omitted from the reaction medium.

Ideally, the reaction medium is free of all monovalent cations.

In a preferred embodiment, the reaction medium includes up to 10 mMol of a magnesium or manganese salt, sub Km levels of ATP and an excess of DNA ligase, the medium being buffered to pH 7.5.

Ideally, therefore, the reaction medium contains 10 mMol MgOAc₂, 10 mMol TrisOAc at a pH of 7.5, 10 μM ATP and 0.5 U/ml T4 DNA ligase.

The present inventor has found that using the reaction medium of the present invention it is possible to perform the method of nucleic acid amplification described in WO99/49079, the contents of which are incorporated herein by reference, efficiently for RNA.s

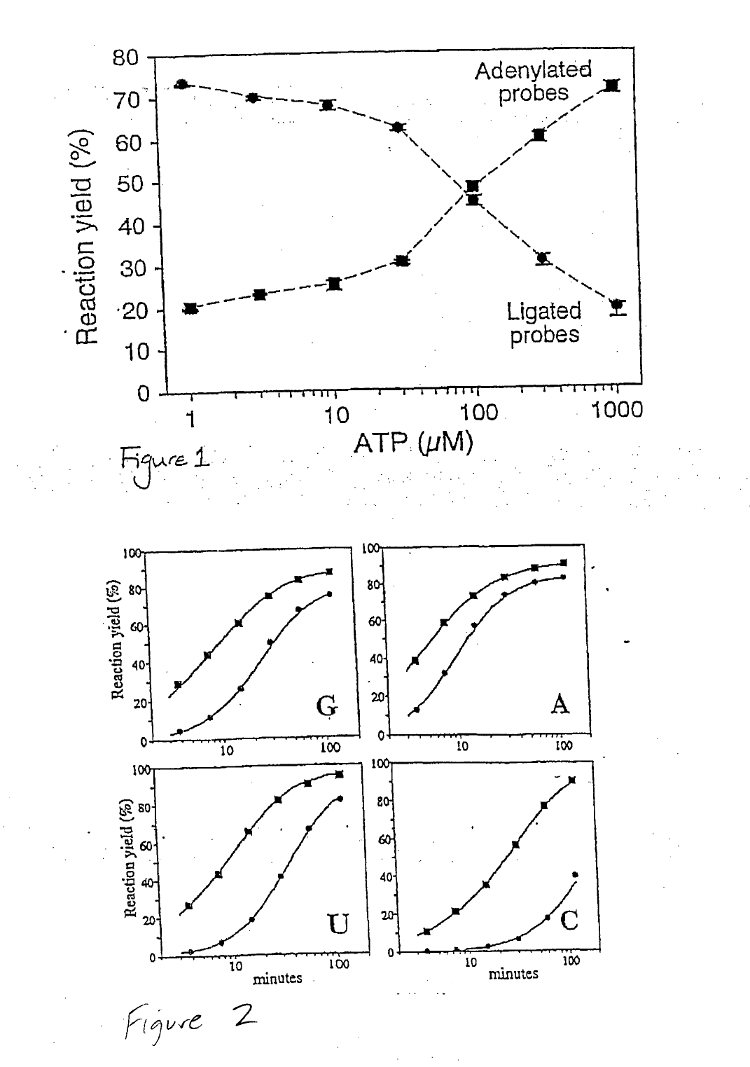

In order to determine if probe ligation reactions are usable to distinguish RNA sequence variants, the present inventors prepared a set of four in vitro transcripts of amplified synthetic oligonucleotides that differed in one centrally located position. They compared the ability of four oligonucleotides to ligate to the 5′ end of a fluorescence-labelled oligonucleotide in separate reactions containing either of the four target variants. The four probe oligonucleotides each had different nucleotides at their 3′ terminal positions, hybridising opposite to a variable position in the target RNA. The different ligation products could be distinguished and quantitated since the four ligation probes, having different 3′ ends, each had a different size sequence addition at the 5′ ends; see FIG. 1 which show the ligation probe oligonucleotides used in the study. Ligation and adenylation of probe molecules were analysed by measuring the area under the peaks recorded by a fluorescence sequencer. The four 5′-probes differ at the ultimate 3′-end and they are identified by their different lengths. 5′ adenylated products of the fluorescent probes migrate with a 1.5 nucleotide greater apparent size compared to the unreacted probe, while each of the four possible ligation products could be distinguished according to their different sizes.

The following description describes conditions which allow efficient ligation of pairs of DNA oligonucleotides, hybridising next to one another on an RNA strand.

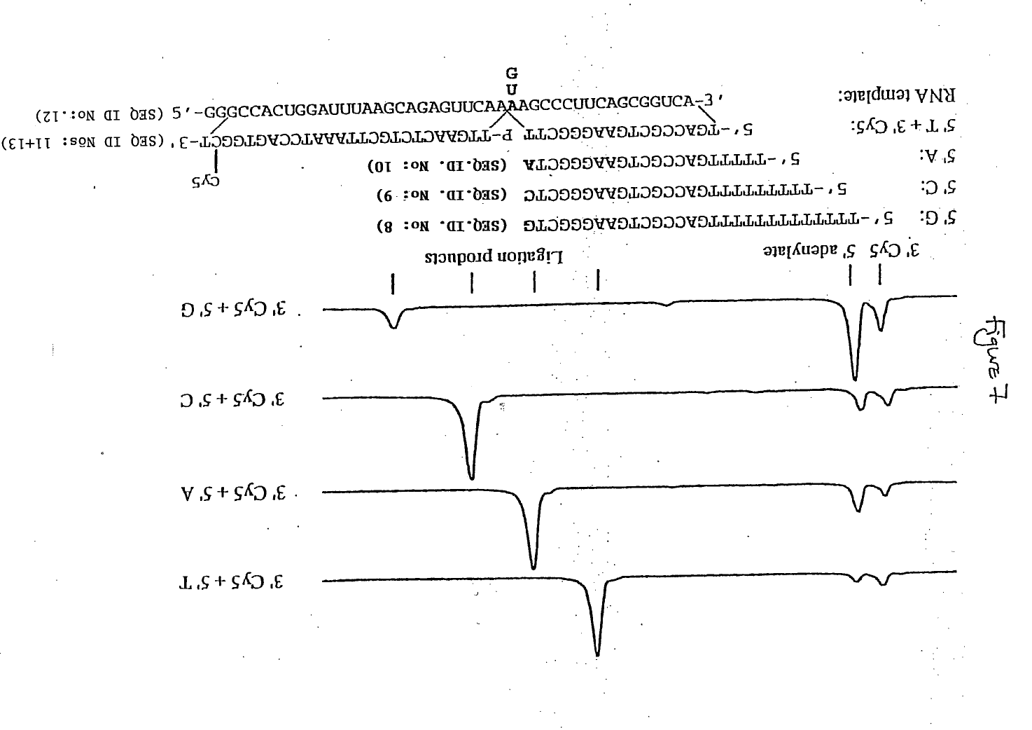

An important advantage of the present invention is that under one standard set of reaction conditions the probe ligation reactions allow distinction of any single nucleotide probe-target mismatch by a factor of between 20- and 200-fold, compared to the corresponding matched probe-target hybrids (see FIG. 5). A further advantage is that the mechanism of the present invention allows padlock probes to be used to distinguish single-nucleotide variants in RNA (see FIG. 6).

Ligase-mediated gene detection according to the invention therefore provides highly sensitive and accurate ligase-mediated detection and distinction of RNA sequence variants in solution, on DNA microarrays, and in situ.

The reaction mechanism of the T4 DNA ligase-catalysed sealing of nicked DNA substrates is known in some detail. (Engler, M. J. and Richardson, C. C. DNA ligases The Enzymes XV 3-29 (1982), Higgins, N. P. and Cozzarelli, N. R. DNA-joining enzymes: a review Methods Enzymol. 68 50-71 (1979), Rossi, R., et al. Functional characterization of the T4 DNA ligase: a new insight into the mechanism of action Nucl. Acids Res. 25 2106-2113 (1997)). The enzyme is first activated through ATP hydrolysis, resulting in the covalent addition of an AMP-group to the enzyme. After recognition of a nicked site in a DNA duplex, the ligase transfers this AMP to the phosphorylated 5′ end at a nick, forming a 5′-5′ pyrophosphate bond. Finally, the ligase catalyses an attack on this pyrophosphate bond by the 3′ end at the nick, thereby sealing the nick, whereafter ligase and AMP are released. However, if the ligase detaches from the substrate before the 3′ attack, e.g. because of premature AMP reloading of the enzyme, then the 5′ AMP will be left at the 5′ end, blocking further ligation attempts.

Rossi et al. propose a model for ligation reactions that involves two different ligase-binding complexes (Rossi, R., et al. Functional characterization of the T4 DNA ligase: a new insight into the mechanism of action Nucl. Acids Res. 25 2106-2113 (1997)). According to their model, a transient complex is formed by the adenylated enzyme that scans the DNA duplex for substrates. The deadenylated enzyme then forms a stable complex when it has transferred its AMP residue to the 5′ phosphate of the substrate.

The stable binding of deadenylated enzyme facilitates the joining reaction by permitting the 3′ end to attack the pyrophosphate bond between the AMP and the 5′ phosphate. The model predicts that the joining of “difficult” substrates, e.g. blunt-end ligation, may be inhibited by premature AMP reloading of the ligase, resulting in dissociation of the enzyme after the 5′ adenylation step (Rossi, R., et al. Functional characterization of the T4 DNA ligase: a new insight into the mechanism of action Nucl. Acids Res. 25 2106-2113 (1997)). Due to the slow kinetics of the DNA joining reaction on RNA targets, the reaction can be predicted from Rossi's model to be inhibited by ATP concentrations exceeding the Km for AMP binding. FIG. 1 shows the ATP dependence of the probe adenylation and ligation reactions on RNA targets. Adenylation and ligation yields after a 60 minute reaction at four different ATP concentrations are shown relative to the highest yield of adenylation and ligation in each ATP titration series. An ATP titration experiment in RNA-templated ligation reactions indeed supports their model, since the yield of adenylated end products increases with increasing ATP concentration, while as a consequence less ligated end products are obtained.

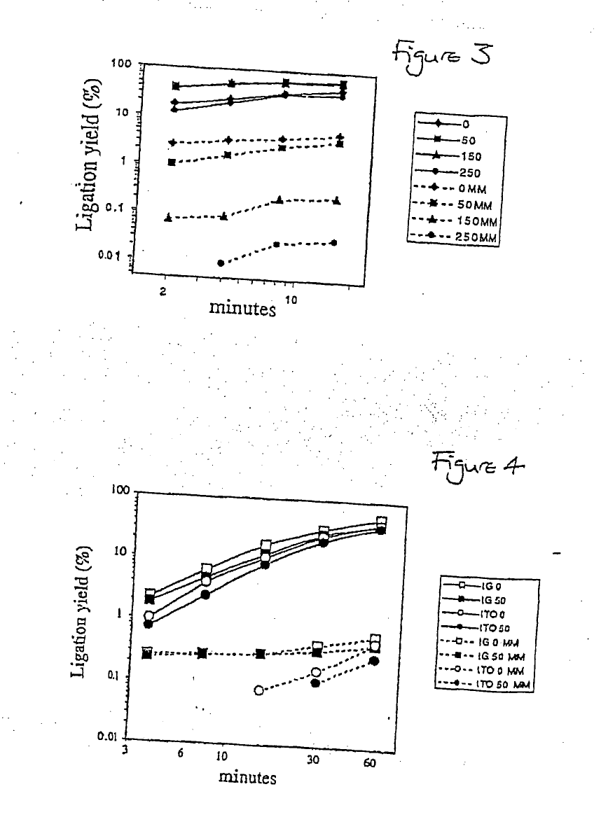

Although the process of DNA ligation can be divided into a series of discrete reaction steps (Engler, M. J. and Richardson, C. C. DNA ligases The Enzymes XV 3-29 (1982),Higgins, N. P. and Cozzarelli, N. R. DNA-joining enzymes: a review Methods Enzymol. 68 50-71 (1979),Rossi, R., et al. Functional characterization of the T4 DNA ligase: a new insight into the mechanism of action Nucl. Acids Res. 25 2106-2113 (1997)), ligation reactions on DNA substrates follow first order kinetics, indicating that only one step in the overall reaction is rate limiting. Under conditions where the enzyme is turned over, and the ATP concentration is above the Km for AMP binding, substrate binding is the limiting reaction step (Engler, M. J. and Richardson, C. C. DNA ligases The Enzymes XV 3-29 (1982),Higgins, N. P. and Cozzarelli, N. R. DNA-joining enzymes: a review Methods Enzymol. 68 50-71 (1979),Rossi, R., et al. Functional characterization of the T4 DNA ligase: a new insight into the mechanism of action Nucl. Acids Res. 25 2106-2113 (1997)). The result from DNA probe-ligation reactions on RNA targets at excess of ligase follow quite different kinetics. FIG. 2 illustrates the time course of ligation of DNA probes, correctly base-paired to four different RNA targets. Aliquots were withdrawn at different time points during four different reactions, each including one of the four RNA targets and a matched probe pair, and adenylation and ligation of yields were determined. In the drawing, Squares represent probes that have been either adenylated or ligated while circles represent ligated probes.

The accumulation of products that have been processed by T4 DNA ligase to form either adenylated or ligated reaction products follow first order kinetics, whereas completed ligation alone does not. This indicates that both the 5′ adenylation step and the joining step are rate limiting. Moreover, the ligation rates differ substantially between the four templates used in the present invention.

A ligase titration experiment suggests that the enzyme is not turned over in the reaction, since it reaches saturation only if an excess of enzyme over substrate is added (data not shown). The initial rate (≈Vmax) of DNA ligation on DNA targets using the T4 DNA ligase has been estimated at 5 turn-overs/second (Tong, J., Cao, W. and Barany, F. Biochemical properties of a high fidelity DNA ligase from Thermus species AK16D Nucleic Acids Res. 27 788-794 (1999)). It is not meaningful to measure the DNA ligation rate on RNA in turn-over numbers. Instead the time required to process half the substrates (t½) can be used as a measure of the reaction rate. The t½ for the complete ligation reaction ranged from 10-150 min in the presence of the four different targets, and it thus proceeds at least 3000 times slower on RNA targets compared to DNA targets.

Ligase-assisted gene detection assays efficiently distinguish between DNA sequence variants, including ones involving single nucleotide differences, due to the strict requirement by some DNA ligases for correctly base-paired substrates. It is well established that the ability of T4 DNA ligase to discriminate mismatches is increased at elevated concentrations of NaCl (Landegren, U., et al. A ligase-mediated gene detection technique Science 241 1077-1080 (1988),Wu, D. Y. and Wallace, R. B. Specificity of the nick-closing activity of bacteriophage T4 DNA ligase Gene 76 245-254 (1989)). The present inventors compared the ability of the two oligonucleotides with 3′ C or T to ligate to a downstream oligonucleotide when hybridised to a DNA variant of the target molecule having a G in the variable position at four different NaCl concentrations. FIG. 3 shows the time course of T4 DNA ligation of the 5′C and 5′T probes on the G DNA target at four different concentrations of NaCl. Match and mismatch ligation data points are connected by solid and broken lines, respectively. The different NaCl concentrations used in the respective experiment are represented by diamonds, squares, triangles and circles for 0, 50, 150 and 250 mM NaCl respectively. The optimal NaCl concentration for the matched oligonucleotide ligation reactions on DNA targets is between 50 and 150 mM NaCl. At these NaCl concentrations the reaction proceeds with an initial velocity of approximately 7 turn-overs/sec, while the ligation reaction is 3 times slower at 0 or 250mM NaCl. In contrast the mismatch ligation reaction is dramatically decreased by NaCl addition. After a 8 min reaction the ratio of ligation of the matched substrate versus the mismatched one increased from less than 10 at 0 mM NaCl to more than 4000 at 250 mM NaCl (FIG. 3). By way of comparison, in the same experimental set-up the match/mismatch ratio for the Tth DNA ligase was 250 (data not shown), slightly lower than the previously reported values of 450 and 840 (Tong, J., et al. Biochemical properties of a high fidelity DNA ligase from Thermus species AK16D Nucleic Acids Res. 27 788-794 (1999),Luo, J., Bergstrom, D. E. and Barany, F. Improving the fidelity of Thermus thermophilus DNA ligase Nucleic Acids Res. 24 3071-3078 (1996)), probably due to differences in experimental conditions.

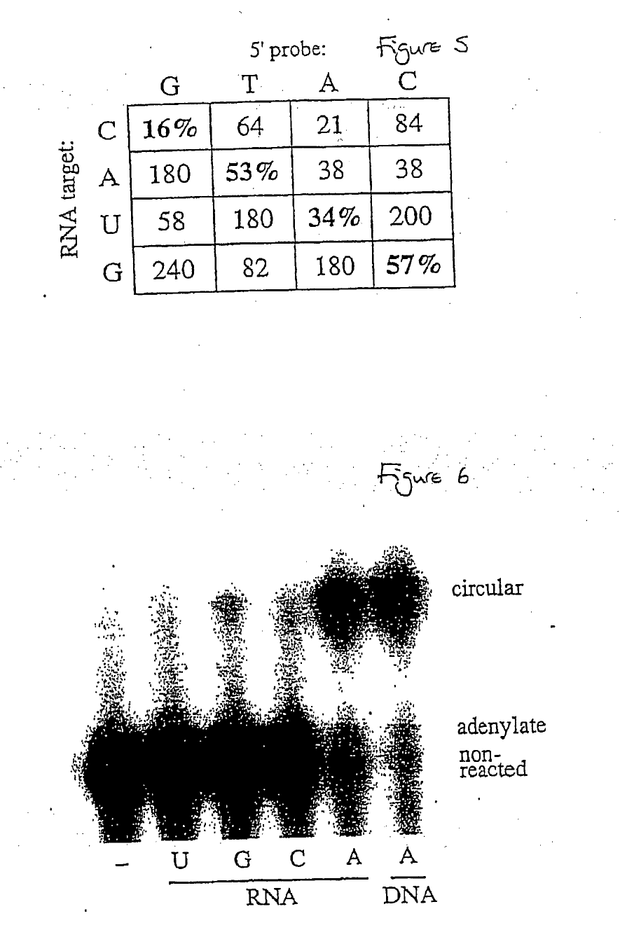

In a similar experiment the present inventors investigated the effect of NaCl concentration on ligation efficiency and distinction of RNA target sequence variants, by probing G and U target sequences with mixes of 3′ C and 3′ T probes and with 3′ A and 3′ G probes, respectively, at two NaCl concentrations. See FIG. 4 which shows the time course of ligation reactions of the 5′C and 5′A probes on each of either the G or the U target RNAs at two different NaCl concentrations. Match and mismatch ligation data points are connected by solid and broken lines, respectively. The results from ligation reactions on the G and U RNA targets are denoted by squares and circles, respectively. The 0 mM and 50 mM NaCl additions to the reactions are represented by open and closed symbols, respectively. After a 30 min ligation reaction at 0 mM NaCl, the A-G and G-U mismatches can be discriminated by a factor of 80 and 150 from the corresponding matched probe and target pairs. The ligase fidelity is only modestly increased by NaCl addition while the ligation reaction is slowed down considerably. Therefore addition of NaCl seems to be of limited value to enhance sequence discrimination of RNA sequence variants. Addition of 150 or 250 mM NaCl completely inhibited both the 5′ adenylation and the joining reaction (data not shown).

There is therefore a need in the art for an ability to be able to improve the efficient detection of RNA targets by ligation.

The present invention allows for efficient joining by T4 DNA ligase of oligonucleotides, hybridising to in vitro transcribed RNA target molecules. Both the 5′ adenylation and the joining step of the ligation reaction proceed considerably slower than on the corresponding DNA targets when conducted at low levels of ATP and NaCl. However, under such conditions RNA targets can be efficiently detected by ligation and using the method of the present invention it is possible to reach an efficiency of detection of target RNA strands of between 75 and 85%, typically of about 80%.

It has been found by the present inventors that the optimal concentration of ATP for RNA ligation is dramatically different (two orders of magnitude or more different) than that for ligation of DNA.

The present inventors have demonstrated that RNA target molecules can be efficiently detected via ligation of oligonucleotides by T4 DNA ligase, provided that the concentrations of both NaCl and of ATP are kept low, that a molar excess of ligase over substrate is used, and that the reaction is given sufficient time. In most biological samples the concentration of any specific RNA sequence is low enough that sufficient T4 DNA ligase can be added to detection reactions. A potentially greater problem is manifested in the considerable difference among the four closely similar RNA target sequences used in this study with respect to ligation kinetics. The reason for this difference is not known. Nonetheless, by using the set of reaction conditions reported here we have shown that all mismatched RNA targets can be clearly distinguished from the corresponding matched ones.

Ligase-based detection of RNA sequence variants should be of value in a number of situations. Applied as a ligase-mediated circularisation of padlock probes, the reaction products can be detected via a rolling-circle replication mechanism, resulting in the synthesis of a long DNA strand composed of hundreds or thousands of copies of the circularised probe (Baner, J., et al. Signal amplification of padlock probes by rolling circle replication Nucleic Acids Res. 22 5073-5078 (1998), Fire, A. and Xu, S.-Q. Rolling replication of short DNA circles Proc. Natl. Acad. Sci. USA 92 4641-4645 (1995),Liu, D., et al. Rolling circle DNA synthesis: small circular oligonucleotides as efficient templates for DNA polymerases J. Am. Chem. Soc. 118 1587-1594 (1996),Lizardi, P. M., et al. Mutation detection and single-molecule counting using isothermal rolling-circle amplification Nature Genet. 19 225-232 (1998)), or even faster amplification can result via the so-called hyperbranched rolling circle amplification mechanism (Lizardi, P. M., et al. Mutation detection and single-molecule counting using isothermal rolling-circle amplification Nature Genet 19 225-232 (1998)). Ligation of probes hybridised to RNA target molecules will permit in situ detection of variants of RNA sequences, and the same mechanism could greatly improve both sensitivity and sequence specificity in quantitative studies of gene expression.

It is known that circularised padlock probes which hybridise to a target nucleic-acid strand are poor substrates for subsequent amplification methods like PCR and RCR (Banér, J., et al. Signal amplification of padlock probes by rolling circle replication Nucleic Acids Res. 22 5073-5078 (1998). This may be due to steric hindrance caused by the target strand that is threaded through the circular probe as many times as the turns in the target-probe heteroduplex.

In a further aspect, the present invention seeks to prime polymerisation and avoid target strand (RNA) inhibition of probe amplification, both for in vitro and in situ amplification of circularised probes.

In this aspect, the present invention provides a method for priming polymerisation of probe amplification in vitro the method comprising the further step after a probe hybridisation and ligation of degrading the target RNA by heating the sample in the presence of magnesium chloride.

This will degrade any RNA present in the sample. As a consequence the probe will be released from its target strand. The polymerisation may then be primed by a DNA primer complementary to the probe.

Alternatively, after hybridisation, the target RNA may be degraded by RNase A.

This enzyme activity will degrade any free RNA present in the sample. However, the probe-target DNA/RNA heteroduplex will remain intact. The remaining RNA sequence may serve as a primer for a subsequent DNA polymerisation reaction. In a further alternative, a DNA primer, complementary to the probe, may be added. The DNA polymerase may then be able to displace the DNA/RNA heteroduplex.

In another aspect, after hybridisation, the target RNA may be degraded by RNase H.

This activity will only degrade the RNA strand in the target-probe RNA/DNA heteroduplex. The degradation is non-processive. As a consequence the first product of the degradation reaction is a single nick in the RNA/DNA heteroduplex. This nick could serve as a primer for a suitable DNA polymerase. Using a balanced mix of RNase H and a DNA polymerase, the RNA strand could initiate the DNA polymerisation reaction. Alternatively, a DNA primer, complementary to the probe, may be added to probes that have been completely released from its target RNA strand.

In yet another aspect, the invention provides for the priming of polymerisation and avoidance of target strand (RNA) inhibition of probe amplification for in situ amplification of circularised probes by using RCR. RCR is specially well suited for in situ amplification of circularised probes, because it is a isothermal amplification and the polymerisation product will be much greater in size than any of the reagent used in the amplification reaction and may therefore remain at the site of the target RNA recognition.

Alternatively, after probe hybridisation and ligation the target RNA may be degraded by RNase H. The RNase H degradation/priming strategy may be particularly well suited for this application, because the RCR product will become an extension of the RNA sequence that has been recognized by the probe. The signal generated by the RCR will by definition be localized at the same spot as the RNA molecule was fixed to the cell matrix.

In another method, after probe hybridisation and ligation the target RNA may be degraded by RNase A. The RNase A strategy might also be useful, because the enzyme could be added in RCR reaction mix and therefore the RCR could be initiated at the very same moment as the probe becomes released from the target RNA strand. This would minimize probe/RCR-product diffusion before the RCR product will obtain the required size to become immobilized in the cellular matrix.

In this respect, in a final aspect the present invention allows for a high through-put format for mRNA isolation and identification of many transcript in many samples involving oligo-dT coated manifold supports, multiplexed padlock probing, followed by RCR amplification, using any of the methods of the present invention described above.

Embodiments of the present invention will now be described by way of example only, with reference to the following drawings, of which:

FIGS. 1 to 4 have already been discussed;

FIG. 5 is a table showing the ligation efficiency and discrimination among all 16 possible single-nucleotides matches and mismatches at the adjoining 3′ end of pairs of DNA probes hybridised to RNA targets;

FIG. 6 is a photograph of a gel showing circularisation of the padlock probes, and

FIG. 7 has already been discussed, and shows SEQ. ID, Nos. 8 to 12 respectively.

Ligation reaction products were analysed by gel electrophoresis in a fluorescence sequencing instrument, to identify and quantify the five modified forms of the fluorescence-labelled 5′ phosphorylated oligonucleotide that can arise in the ligation reactions. One of these reaction products is the 5′ adenylated form of the probe, which is formed as an intermediary product during the ligation reaction, while the remaining four products represent completed joining of each of the size-coded probe oligonucleotides to the fluorescence-labelled one.

In the ligation of DNA probes hybridised to RNA targets, NaCl concentrations above 50 mM seriously inhibited the reaction, while ligation efficiency and mismatch discrimination was similar at 0 and 50 mM NaCl. Therefore no NaCl was added to RNA-templated ligation reactions. For each of the four correctly matched probe-target combinations, displayed along the diagonal in FIG. 5, the percentage of labelled oligonucleotides that were ligated in the presence of an excess of RNA target molecules is shown. FIG. 5 shows the ligation efficiency and discrimination among all 16 possible single-nucleotide matches and mismatches at the adjoining 3′ end of pairs of DNA probes hybridised to RNA targets. For all four target sequence variants, the percentage of correctly matched oligonucleotides that were ligated in a 90 minute reaction is presented in bold. The relative yield of each of the three mismatched probes are given as a ratio of the ligation of the corresponding correctly matched oligonucleotide, present in the same reaction. The ligation of each of the 12 possible mismatched substrates are presented as the ratios between the ligation efficiency of each reaction and that of the corresponding matched reaction. Most mismatches are discriminated by a factor greater than 80, compared to the corresponding matched substrate (G-G, T-G, A-G, T-U, C-U, G-A, C-C). All the remaining substrates are discriminated by greater than 20-fold (G-U, C-A, T-C, A-C, A-A), adequate for robust distinction among RNA sequence variants.

Circularisable oligonucleotides—padlock probes—are linear oligonucleotide probes with 5′ and the 3′ segments complementary to immediately adjacent target sequences. Upon hybridisation to target molecules the probes can be converted to circular oligonucleotides by ligation. The target-dependent circularisation of padlock probe has been used to distinguish single-nucleotide variants of DNA sequences in metaphase preparations, and for mutation detection in human genomic DNA. Circularisable DNA probes have also been put to use for detection of RNA sequences, followed by signal amplification via a combined PCR and rolling-circle replication reaction. In order to determine whether such probes will be useful to detect single-nucleotide variants of RNA sequences, we constructed a probe with target-complementary sequences at the 5′ and 3′ ends, corresponding to the probe pair having a T at the variable 3′ position in the previous experiments. FIG. 6 demonstrates that efficient ligation was only observed in the presence of the target RNA with an A in the position opposite the 3′ end of the padlock probe, thus distinguishing all three mismatches from the matched substrate. FIG. 6 shows that ligase-mediated circularisation of padlock probes is sensitive to single-nucleotide mismatches at the probe's 3′ end. A padlock probe designed to specifically circularise in the presence of the A variant of the target sequences was hybridised to the four different RNA sequences and the DNA version of the A variant and then incubated together with T4 DNA ligase for 30 minutes. The negative control (−) did not contain any target sequence.

These results demonstrate that variants of RNA target molecules can be efficiently detected via ligation of oligonucleotides by T4 DNA ligase. Typically around 80% of probes hybridised to a matched RNA target molecule are ligated under the reaction conditions we describe. As seen in FIG. 3 there is, however, a considerable difference among the four closely similar RNA target sequences used in this study with respect to ligation efficiency. The reason for this difference is not known. Nonetheless, all mismatched RNA targets were clearly distinguished from the corresponding matched ones.

Ligase-based detection of RNA sequence variants should be of value in a number of situations. Applied as padlock probes, the ligation products can be detected via a rolling-circle replication mechanism, resulting in the synthesis of a long DNA strand composed of hundreds or thousands of copies of the circularised probe, or even faster amplification can result via the so-called hyperbranched rolling circle amplification mechanism. Ligation of probes hybridised to RNA target molecules will permit in situ detection of variants of RNA sequences, and the same mechanism could greatly improve both sensitivity and sequence specificity in quantitative studies of gene expression.

Experimental Protocol

Oligonucleotides. Oligonucleotides used as ligation probes (FIG. 1.) were purified by reversed phase chromatography (RP-18, Pharmacia Biotech) both before and after detritylation. One oligonucleotide, chemically 5′-phosphorylated at the conclusion of oligonucleotide synthesis 19, included a C-residue modified with a primary amine (Sund, C., Ylikosli, J., Hurskainen, P. & Kwiatkowski, M. Construction of europium (Eu3+)-labelled oligo DNA hybridisation probes. Nucleos. Nucleot. 7, 655-659 (1988)). After synthesis a Cy5-fluorophore was conjugated to this primary amine using a Cy5-N-hydroxysuccinimide ester (Pharmacia Biotech), and the reagent was purified as above. The padlock probe (5′ P-TTGAACTCTGCTTAAATCCAGTGGTTTTTTTTTTTTTTTATGTTAAGTGACC GGCAGCATTTTTTTTTTTTTTTTGACCGCTGMGGGCTT -3′) (SEQ. ID No: 1) was synthesized, purified, and finally radioactively labelled according to Kwiatkowski et al (Kwiatkowski M., Nilsson, M. & Landegren, U. Synthesis of full-length oligonucleotides: cleavage of apurinic molecules on a novel support. Nucleic Acids Res. 24, 4632-4638 (1996).) Synthesis of RNA ligation templates. Templates for in vitro transcription were synthesized by PCR, using as amplification templates the four oligonucleotides 5′-CCACTG-GATTTAA-GCAGAG-TTCAAN-AGCCCTTC-AGCGG-TCA-3′, (SEQ. ID No: 2) where N represents one of the four nucleotides. One fmol of each amplification template was combined in four separate reactions with 0.2 μM of the primers 5′-AATTTA-ATACGA-CTCACTAT-AGGGCCAC-TGGATTTAAG-CAGAG-TTCA-3′, (SEQ.ID No. 3) having a T7 promoter sequence added at the 5′ end 22, and 5′-biotin-TGACCGCTGAAGGGC-3′, (SEQ.ID No. 4) and Taq polymerase in 100 μl of 50 mM KCl, 10 mM Tris-HCl pH 8.3, 1.5 mM MgCl2, 12.5 μg/ml BSA, and 200 μM dNTP. The reactions were thermally cycled 20 times between 94° C., 55° C., and 72° C., 30 seconds each, followed by seven min at 72° C. Amplification products from ten μl of each PCR were bound to 20 μl of washed paramagnetic streptavidin-coated beads in 100 μl 1M NaCl, 50 mM Tris-HCl pH 7.0, at room temperature for one hour under constant rotation. The beads were washed three times in binding solution, once in deionised sterile water, and once in transcription buffer (40 mM Tris-HCl pH 7.5, 6 mM MgCl2, 2 mM spermidine, and 10 mM NaCl). In vitro transcription was performed in 100 μl transcription buffer with 10 mM dithiothreitol, 0.5 mM NTPs, 150 U human placental ribonuclease inhibitor (Amersham), and 50 U of T7 RNA polymerase (Stratagene) at 37° C. for one hour during constant rotation of the tubes. After removal of beads, the in vitro transcripts were centrifuged twice through Sephadex G-50 Nick Columns (Pharmacia Biotech) to remove salts and nucleotides. We estimate that the DNA content of the in vitro transcribed RNA is less than one in a thousand RNA molecules by comparing the results from PCR of in vitro transcription reactions with a dilution series of transcription templates (data not shown).

Ligation reactions on RNA templates. Ligation reactions were performed in 10 mM MgOAc₂, 10 mM TrisOAc pH 7.5, 10 μM ATP, and 0.5 U/μl T4 DNA ligase (Amersham Pharmacia Biotech) at 37° C. for up to four hours. In order to preform 40 nM nicked heteroduplex molecules prior to the addition of ligase, ligation probes and the RNA templates were added to ligation mixes at a molar ratio of 1:2:4 (Cy5 labelled 3′ oligonucleotide:RNA target:5′ oligonucleotide). The ligation mixes were incubated at 65° C. for 3 min, and subsequently slowly cooled to room temperature, and finally placed on ice. The ligation reactions were terminated by adding an equal volume of stop buffer (95% formamide, 25 mM EDTA, and Dextran blue) to the reaction, or alternatively, in time-course reactions, 5 μl of the reaction were added to 5 μl of stop buffer. To avoid renaturation of probes and targets, which results in extra bands during electrophoresis, one μl of 1M NaOH was added to the terminated reactions, and these were incubated at 65° C. for 15 min to degrade the RNA target.

Analysis of ligation products. Products of the ligation reactions were analysed using an ALF Express sequencing instrument (Amersham Pharmacia Biotech) with a 7M urea, 10% polyacrylamide gel in 0.6×TBE buffer. The instrument has a linear dynamic range of fluorescence detection over an at least thousand-fold concentration range according to the manufacturer and our own measurements (data not shown). The fluorescence was recorded in real-time and processed using software developed for the instrument (AlleleLinks).

Padlock probe circularisation on RNA and DNA targets. The ligation reactions on both RNA and DNA targets were performed for 30 minutes using the same conditions as above for ligation on RNA targets. The reactions products were separated on a denaturing 6% polyacrylamide gel, which was subsequently scanned and analysed using a Phosphorlmager (Molecular Dynamics).

Oligonucleotides to be used as ligation probes (FIG. 7) were synthesized on an ABI 374 oligonucleotide synthesizer, and purified by reversed phase chromatography (RP-18, Pharmacia Biotech) both before and after detritylation. One oligonucleotide was chemically 5′-phosphorylated at the conclusion of oligonucleotide synthesis (Connolly, B. A. Solid phase 5′-phosphorylation of oligonucleotides Tetrahedron Left. 28 463-466 (1987). During synthesis of this oligonucleotide a C-residue modified with a primary amine was incorporated (Sund, C., Ylikosli, J., Hurskainen, P. & Kwiatkowski, M. Construction of europium (Eu3+)-labelled oligo DNA hybridisation probes. Nucleos. Nucleot. 7, 655-659 (1988)). After synthesis a Cy5-fluorophore was conjugated to this primary amine using a Cy5-N-hydroxysuccinimide ester (Pharmacia Biotech), and the reagent was purified as above. PCR primers and oligonucleotides used as DNA ligation templates and as templates for PCR were purchased from Interactiva (Ulm, Germany).

Synthesis of RNA ligation templates. Templates for in vitro transcription were synthesized by PCR using as amplification templates the four oligonucleotides 5′-CCACTG-GATTTAA-GCAGAG-TTCAAN-AGCCCTTC-AGCGG-TCA-3′, (SEQ. ID. No: 5) where N in each oligonucleotide represents one of the four nucleotides. One fmol of each amplification template was combined in four separate reactions with 0.2 μM of the primers 5′-AATTTA-ATACGA-CTCACTAT-AGGGCCAC-TGGATTTAAG-CAGAG-TTCA-3′ (SEQ.ID No: 6) having a T7 promoter sequence added at the 5′ end [Fahy, 1991#553] and 5′-Biotin-TGACCGCTGAA-3′, (SEQ.ID. No. 7) and Taq polymerase in 100 μl of 50 mM KCl, 10 mM Tris-HCl pH 8.3, 1.5 mM MgCl2, 12.5 μg/ml BSA, and 200 μM dNTP. The reactions were thermally cycled 20 times between 94° C., 55° C., and 72° C., 30 seconds each, followed by seven min at 72° C. Amplification products from 10 μl of each PCR were bound to 20 μl of washed paramagnetic streptavidin-coated beads, suspended in a binding solution of 100 μl 1M NaCl, 50 mM Tris-HCl pH 7.0, at room temperature for one hour. The tubes were kept under constant rotation to avoid sedimentation of the beads. The beads were washed three times in binding solution, once in deionised sterile water, and once in transcription buffer (40 mM Tris-HCl pH 7.5, 6 mM MgCl2, 2 mM spermidine, and 10 mM NaCl). In vitro transcription was performed in 100 μl transcription buffer with 10 mM dithiothreitol (DTT), 0.5 mM NTPs, 150 U human placental ribonuclease inhibitor (Amersham), and 50 U of T7 RNA polymerase (Stratagene) at 37° C. for one hour during constant rotation of the tubes. After removal of beads, the in vitro transcripts were centrifuged twice through Sephadex G-50 Nick Columns (Pharmacia Biotech) to remove salts and nucleotides. We estimate that the DNA content of the in vitro transcribed RNA is less than one in a thousand RNA molecules by comparing the results from PCR of in vitro transcription reactions with a dilution series of transcription templates (data not shown).

Ligation reactions on RNA templates. If not otherwise indicated ligation reactions were performed in 10 mM MgOAc_{2,} 10 mM TrisOAc pH 7.5, 10 μM ATP, and 0.5 U/μl T4 DNA ligase (Amersham Pharmacia Biotech) at 37° C. for up to four hours. In order to preform 40 nM nicked heteroduplex molecules prior to the addition of ligase, ligation probes and the RNA templates were added to ligation mixes at a molar ratio of 1:2:4 (Cy5 labelled 3′ oligonucleotide:RNA target:5′ oligonucleotide). The ligation mixes were incubated at 65° C. for 3 min, and subsequently slowly cooled to room temperature, and finally placed on ice. The ligation reactions were terminated by adding an equal volume of stop buffer (95% formamide, 25 mM EDTA, and Dextran blue) to the reaction, or alternatively, in time-course reactions, 5 μl of the reaction were added to 5 μl of stop buffer. To avoid renaturation of probes and targets which results in extra bands during electrophoresis, one pl of 1 M NaOH was added to the terminated reactions, and these were incubated at 65° C. for 15 min to degrade the RNA target.

Ligation reactions on DNA templates. Ligation reactions were performed as above in 10 mM MgOAc, 10 mM TrisOAc pH 7.5, 250 mM NaCl, 1 mM ATP, and 0.04 mU/μl T4 DNA ligase at 37° C. for up to 16 min. Ligation reactions using Tth ligase were performed in 10 mM Tris-HCl pH 7.9, 10 mM MgCl2, 100 mM KCl, 0.1% Triton X-100, 10 mM DTT, and 0.05 U/μl Tth ligase (a kind gift from Dr. Francis Barany) at 55° C. for up to 16 min. Reactions were terminated as above, but no NaOH was added prior to gel analysis.

Analysis of ligation products. Products of the ligation reactions were analysed using an ALF Express sequencing instrument (Amersham Pharmacia Biotech) with a 7M urea, 10% polyacrylamide gel in 0.6×TBE buffer. The instrument has a linear dynamic range of fluorescence detection over at least 103 according to the manufacturer and our own measurements (data not shown). The fluorescence was recorded in real-time and processed using software developed for the instrument (AlleleLinks).

1-7. (canceled)

8. A method of detecting a target sequence of a target RNA characterized in that the method comprises the steps of:
i) providing first and second DNA oligonucleotide probes complementary to a region of said RNA such that the first and second DNA oligonucleotide probes hybridize to the RNA so that the ends of the oligonucleotides are juxtaposed,
ii) forming a hybrid of the first and second DNA oligonucleotide probes with the target RNA and ligating the ends of the first and second DNA oligonucleotide probes to form a ligation product, wherein the ligation step is performed in the presence of less than 50 mM monovalent cations and a concentration of ATP that is less than or equal to the Km for ligase adenylation,
iii) degrading the target RNA at or near the target sequence, and
iv) detecting the presence of the ligation product, wherein the presence of the ligation product is indicative of the presence of the target RNA.

9. The method of claim 8, wherein the concentration of ATP in the ligation step is between 0.1 and 100 μM.

10. The method of claim 8, wherein the concentration of ATP in the ligation step is 10 μM.

11. The method of claim 8, wherein the concentration of monovalent cations in the ligation step is about 0 mM.

12. The method of claim 8, wherein the concentration of magnesium ions or manganese ions in the ligation step is between 5 and 25 mM.

13. The method of claim 12, wherein the concentration of magnesium ions or manganese ions in the ligation step is between 8 and 15 mM.

14. The method of claim 12, wherein the concentration of magnesium ions in the ligation step is about 10 mM.

15. A method of detecting a target sequence of a target RNA wherein the method comprises the steps of:
i) providing a padlock probe for the target RNA sequence,
ii) forming a hybrid of the padlock probe with the target RNA and ligating the ends of the padlock probe to form a circularized padlock probe in a reaction wherein the ligation step is performed in the presence of less than 50 mM monovalent cations and a concentration of ATP that is less than or equal to the Km for ligase adenylation,
iii) degrading the target RNA at or near the target sequence without degrading the circularized padlock probe, and
iv) effecting rolling circle replication of the padlock probe.

16. The method of claim 15, wherein the concentration of monovalent cations in the ligation step is about 0 mM.

17. The method of claim 15, wherein the concentration of ATP in the ligation step is between 0.1 and 100 μM.

18. The method of claim 17, wherein the concentration of monovalent cations in the ligation step is about 0 mM.

19. The method of claim 15, wherein the concentration of ATP in the ligation step is 10 mM.

20. The method of claim 17, wherein the concentration of magnesium and manganese ions in the ligation step is between 5 and 25 mM.

21. The method of claim 20, wherein the concentration of magnesium ions or manganese ions in the ligation steps is between 8 and 15 mM.

22. The method of claim 20, wherein the concentration of magnesium ions in the ligation step is about 10 mM.

23. The method of claim 15, wherein the step of degrading the target RNA comprises a limited degradation within the target sequence, using enzymatic or chemical activity, to provide a primer to prime rolling circle replication of the padlock probe in step iv).

24. The method of claim 17, wherein the step of degrading the target RNA comprises a limited degradation within the target sequence, using enzymatic or chemical activity, to provide a primer to prime rolling circle replication of the padlock probe in step iv).

25. The method of claim 15, wherein the target RNA is degraded in step iii) using a RNase A-like or chemical activity so that the portion of the target RNA that forms a hybrid with the padlock probe is not degraded and serves as a primer for rolling circle replication of the padlock probe in step iv).

26. The method of claim 17, wherein the target RNA is degraded in step iii) using a RNase A-like or chemical activity so that the portion of the target RNA that forms a hybrid with the padlock probe is not degraded and serves as a primer for rolling circle replication of the padlock probe in step iv).

27. The method of claim 15, wherein the target RNA is degraded using an RNase H activity.

28. The method of claim 17, wherein the target RNA is degraded using an RNase H activity.
